(19) United States
(12) Patent Application Publication
(10) Pub. No.: US 2007/0125959 A1
(43) Pub. Date: Jun. 7, 2007

(54) Open-channel radiation sterilization system

(76) Inventors: Jian Chen, Fujian (CN); Yimin He, Fujian (CN)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/605,613

(22) Filed: Nov. 27, 2006

(30) Foreign Application Priority Data
Dec. 1, 2005 (CN) 200510128434.0

(51) Int. Cl. G01N 21/00

(52) U.S. Cl. 250/453.11

(57) ABSTRACT

An open-channel radiation sterilization system, comprising a channel, a radiation source and an electrical control means, characterized in that the fluid to be sterilized flows in under the radiation source and the sterilized fluid flows out above the radiation source in the channel; a first partition wall and a second partition wall are both provided in the channel, partitioning the channel into three parts, i.e. an inlet zone, a sterilization zone and an outlet zone; the first partition wall is a flow guide wall, which forces the fluid to flow into the sterilization zone via an opening provided at the bottom thereof; the second partition wall is a fluid level control wall, the body and bottom of which are closed against fluid and which forces the fluid passing through the sterilization zone to flow out via an opening provided at the top thereof; the height at the top of the opening provided at the bottom of the flow guide wall is lower than the height at the top of the fluid level control wall, and also lower than the height at the bottom of the radiation source; the height at the top of the flow guide wall is not limited, as long as it is ensured that the fluid flowing in the inlet zone would not get over the top of the flow guide wall and into the sterilization zone.

### FIELD OF THE INVENTION

The invention relates to fluid radiation sterilization, and more particularly to an open-channel radiation sterilization system.

### DESCRIPTION OF RELATED ART

A fluid radiation sterilization system generally refers to a system in which radiation is applied to the fluid by use of ultraviolet (UV) lamps, a neutron source or other radiation sources, so as to annihilate the noxious substance in the fluid.

It is well known that a fluid UV sterilization system requires keeping a certain fluid level so as to ensure the sterilization effect. When the flow rate of the fluid increases such that the fluid level goes beyond the effective radiation distance of the UV sterilization system (said effective radiation distance means the largest distance between a UV light source and viruses or bacteria that can be annihilated or deactivated by the UV light source in the fluid to be sterilized, with the fluid to be sterilized having a certain transmissivity), part of the fluid will be drained away directly without being effectively sterilized, thus impairing the overall sterilization effect. Therefore, it is necessary to control the fluid level or the flow rate of the fluid inflow so that the fluid level will not go beyond the effective radiation distance of UV lamp module(s) towards the fluid surface, while still being not lower than the lowest fluid level limit. Said lowest fluid level limit generally defines the smallest allowable distance between the highheat parts of the UV lamps and the fluid surface, so as to ensure that the highheat parts of the UV lamps would not being exposed to the atmosphere, thereby facilitating the protection of the UV lamps and lengthening their service life as well.

A known technique involves providing at least one overflow gutter on the downstream of the mounting bracket of the UV lamps, near the end of the channel, which is also used as an outlet port of the sterilization system. The main body of the overflow gutter is cuboid-shaped, and the top edge of the gutter is on the same level with the lowest fluid level limit of the system or is slightly lower than the latter. The gutter has openings in one or two of its end portions, which are used as outlet openings communicating with the drainage passage of the sterilization system. When the flow rate increases such that the fluid surface in the channel becomes higher than the top edge of the gutter, the fluid above the top edge will get over said top edge and into the gutter, further flows into the drainage passage of the system via the outlet openings of the gutter, thereby achieving the purpose of keeping the fluid level. In the case where the size and number of said at least one overflow gutter are to be predetermined according to the practical conditions of the system, the larger the flow rate or the variation range of the flow rate is, the more overflow gutters are needed with intervals being provided between adjacent ones, thus the channel has to be lengthened. Such a well-designed overflow gutter arrangement can keep the fluid level reliably without the need of any electrical and accessional devices, thus it is of simple structure and easy for maintenance. Furthermore, it can operate reliably and safely, and consume less energy. However, in the case where the flow rate or the variation range of the flow rate is large, in order to ensure the sterilization effect, it is necessary to provide more overflow gutters for drainage of fluid so that the fluid level would not go beyond the effective radiation distance of the system. Therefore, the channel has to be considerably lengthened and the cost for construction and land has to be increased, and the drainage of fluid is also disadvantageously influenced.

Other techniques involve providing a flap valve or an electrical gate at the end portion of the channel to control the fluid level. Similarly, in this case the highest fluid level in the sterilization area should not go beyond the effective radiation distance of the UV lamp module(s), otherwise, part of the fluid which is beyond the effective radiation distance would flow out of the channel without receiving sufficient UV radiation, thus impairing the overall sterilization effect. Therefore, for such a system adopting any of the above-mentioned three kinds of drainage measures while not using other fluid level control devices at the same time, it is desirable that the highest fluid level should not go beyond the effective radiation distance, which leads to a low difference value of pressure heads between the inlet end and the outlet end of the UV lamp module(s), and a small allowable variation range of the flow rate of the fluid inflow. In this system it is not possible to ensure that all the fluid in the channel would pass through the effective radiation area and receive sufficient UV radiation when the flow rate or the variation range of the flow rate is large. Therefore, a radiation sterilization system with a simple structure and occupying less area is needed, which can control the fluid level automatically and operates reliably, so as to meet the requirement for fluid treatment.

Moreover, the above-mentioned radiation sterilization systems all have the drawback of leading to pressure head loss after the fluid has passed through the radiation source.

### SUMMARY OF THE INVENTION

Therefore, the object of this invention is to provide an open-channel radiation sterilization system with a simple structure and occupying less area, which leads to no pressure head loss, and can keep the fluid level automatically and operate reliably.

According to the technical solution of the present invention, an open-channel radiation sterilization system comprises a channel, a radiation source and an electrical control means, wherein the fluid to be sterilized flows in under the radiation source and the sterilized fluid flows out above the radiation source in the channel; a first partition wall and a second partition wall are both provided in the channel, partitioning the channel into three parts, i.e. an inlet zone, a sterilization zone and an outlet zone; the first partition wall is a flow guide wall, which forces the fluid to flow into the sterilization zone via an opening provided at the bottom thereof; the second partition wall is a fluid level control wall, the body and bottom of which are closed against fluid and which forces the fluid passing through the sterilization zone to flow out via an opening provided at the top thereof; the height at the top of the opening provided at the bottom of the flow guide wall is lower than the height at the top of the fluid level control wall, and also lower than the height at the bottom of the radiation source; the height at the top of the flow guide wall is not limited, as long as it is ensured that the fluid flowing in the inlet zone would not get over the top of the flow guide wall and into the sterilization zone. Said partition walls are perpendicular to, parallel to or at an angle with the general flow direction in the channel.

In order to improve the sterilization effect, a plurality of UV sterilization sections may be provided in the channel along the general flow direction therein, each UV sterilization section is constituted by an inlet zone, a sterilization zone and an outlet zone, and the fluid flowing out of a preceding UV sterilization section enters into the inlet zone of the next UV sterilization section.

The radiation sterilization system according to the present invention has the following advantages:

1. The fluid level in the outlet zone is kept by the fluid level control wall, in other words, the fluid level remains constant as long as the height of the fluid level control wall is set, thus the loss of pressure head in the system can be avoid.

2. There are only a channel, a radiation source and a control device in the system, and the fluid level control of the system is achieved by means of a fluid level control wall on the downstream end of the system. Since no overflow gutter or flap valve or electrical gate is needed, the system is of simple structure and is easy for assembly and maintenance.

3. A flow guide wall is provided on the upstream of the radiation source in the system, guiding the fluid to flow in the sterilization zone under the radiation source, thus increasing the radiation amount that the fluid receives by making the fluid to pass through the whole effective radiation area of the radiation source, improving the sterilization effect and enhancing the operational reliability and safety of the system.

4. No overflow gutter is needed in the system, thus the length of the channel can be shortened, the area occupied by the system can be reduced, the cost for construction and land can be saved, and the arrangement of the system can be simplified.

5. No electrical fluid level control device is needed in the system, thus reducing the power consumption.

6. The fluid overflows out of the system naturally, thus the operation of the system is safe and reliable, and the stability of the system is improved.

7. It is possible to regulate the flow rate by sizing the opening at the bottom of the flow guide wall, so as to improve the sterilization effect.

8. The height of the fluid level control wall may be set in such a manner that the fluid level in the sterilization zone always remains above the top of the radiation source, thus facilitating the protection of the radiation source and lengthening the service life thereof.

### BRIEF DESCRIPTION OF THE DRAWINGS

FIG. 1 is a schematic plan view of the first embodiment according to the present invention;

FIG. 2 is a schematic sectional view of the first embodiment according to the present invention; and

FIG. 3 is a schematic plan view of the second embodiment according to the present invention.

As shown in the figures, a sterilization channel is designated by 1; a UV lamp module is designated by 2; an electrical control box is designated by 3; a flow guide wall is designated by 4; a fluid level control wall is designated by 5; an inlet pipe is designated by 6; an outlet pipe is designated by 7; a channel cover plate is designated by 8; the ground is designated by 9; an opening at the bottom of the fluid guide wall is designated by 10; an opening at the top of the fluid level control wall is designated by 11; an inlet zone is designated by A; a sterilization zone is designated by B; an outlet zone is designated by C; the height of fluid level in the inlet zone A is designated by H1; the height of fluid level in the sterilization zone B is designated by H2; and the height of fluid level in the outlet zone C is designated by H3.

### DETAILED DESCRIPTION OF PREFERRED EMBODIMENT

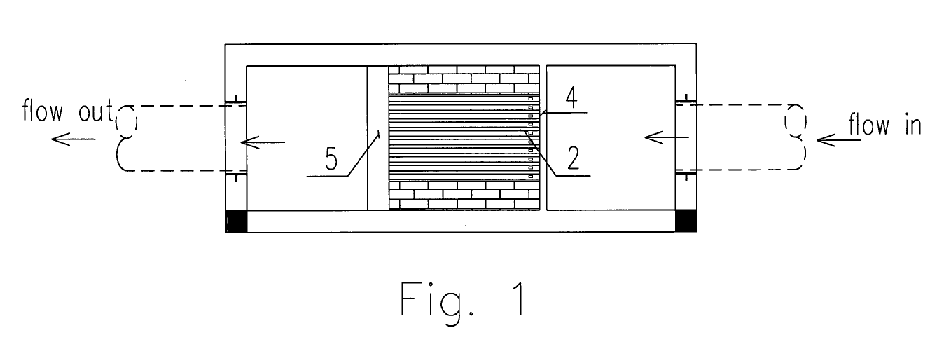

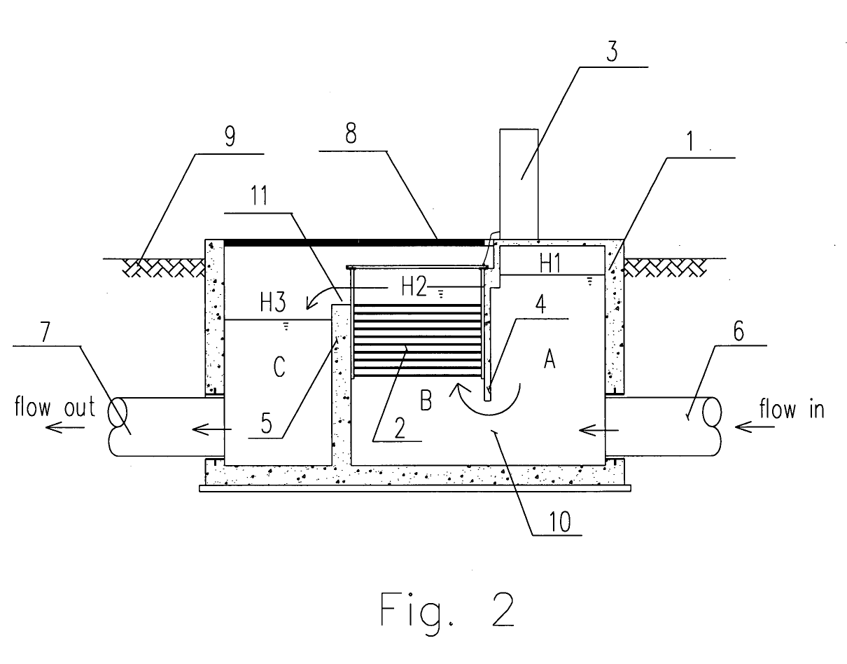

In the first embodiment, as shown in FIGS. 1 and 2, partition walls 4, 5 are respectively provided on the upstream and downstream of a UV lamp module 2 in a sterilization channel 1, partitioning the channel into three parts, i.e. an inlet zone A, a sterilization zone B, and an outlet zone C. The UV lamp module is arranged in the sterilization zone B, which communicates with the inlet zone A and the outlet zone C through the openings 10, 11 respectively formed in the partition walls 4, 5. The partition wall 4 on the upstream of the UV lamp module 2 is a flow guide wall, the body of which is closed against fluid, while near the bottom of the channel 1 there is provided an opening 10 for communicating the fluid. The partition wall 5 on the downstream of the UV lamp module 2 is a fluid level control wall, the body and bottom of which is closed against fluid, while at the top of which there is provided an opening for communicating the fluid and controlling the fluid level in the sterilization zone. The height at the top of the opening 10 provided in the flow guide wall 4 is lower than the height at the top of the fluid level control wall 5. Said partition walls 4, 5 are perpendicular to the general flow direction in the channel. In the present embodiment the UV lamps are parallel to the general flow direction in the channel. The top of the flow guide wall is closed.

Said partition walls 4, 5 may be perpendicular to, parallel to or at an angle with the general flow direction in the channel.

Though the height at the top of the flow guide wall 4 is not limited, measures must be taken to heighten or close it, so as to ensure that the fluid flowing in the inlet zone will not get over the top of the flow guide wall and into the sterilization zone.

In order to improve the heat dispersion effect of the electrical control box, it is possible to settle the box 3 in the inlet zone A or in the outlet zone C, or embed it in the channel 1 or in the partition walls 4, 5, so as to get heat dispersed through the fluid flow.

In order to achieve a better sterilization effect and a better fluid level keeping effect, the size of the opening 10 at the bottom of the flow guide wall 4 and the height at the top of the fluid level control wall 5 may be adjustable.

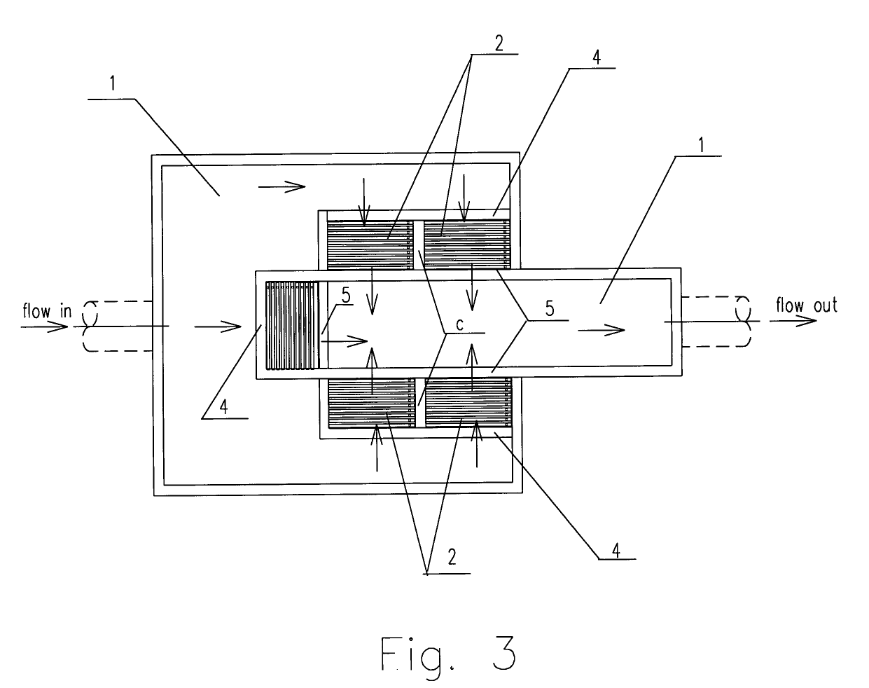

In the second embodiment, as shown in FIG. 3, three UV lamp sterilization modules 2 are provided in the channel 1. On the upstream of each UV sterilization module is provided a flow guide wall 4, while on the downstream of each UV sterilization module is provided a fluid level control wall 5. The function and arrangement of the flow guide wall 4 and the fluid level control wall 5 are the same as in the first embodiment. That is to say, the flow guide wall 4 is provided for fluid inflow, the fluid level control wall 5 is provided for fluid outflow, and the fluid level control wall 5 communicates with the outlet zone of the channel 1. The fluid flows into the channel 1 via an inflow port, and into the UV sterilization zone via the opening at the bottom of the fluid guide wall 4, then the sterilized fluid flows into the outlet zone of the channel via the top of the fluid level control wall 5, and is finally drained away through an outlet pipe. Said partition walls 4, 5 are either perpendicular to or parallel to the general flow direction in the channel. A wall C is provided for mounting the UV lamp modules thereon, and it may be either closed or open at the bottom thereof. Other walls for mounting the UV lamp modules and for constituting the channel are all closed at the bottoms thereof

In the first embodiment, the UV lamp module is perpendicular to the general flow direction in the channel, while in the second embodiment the UV lamp modules are either parallel to or perpendicular to the general flow direction in the channel. In practical applications, the UV lamps may be perpendicular to, parallel to or at an angle with the general flow direction in the channel, or may be a combination of the above-mentioned arrangements.

In the above embodiments the radiation source is UV lamps, but it can also be a neutron radiation source, or other radiation source that can achieve sterilization effect by applying radiation to the fluid.

The open-channel radiation sterilization system according to the present invention can be used for the sterilization of domestic sewage, industrial sewage, recycled water, tap water and other kinds of water.

Various modifications and improvements can be envisaged by those skilled in the art, without departing from the spirit and gist of the invention.

We claim:

1. An open-channel radiation sterilization system, comprising a channel, a radiation source and an electrical control means, characterized in that the fluid to be sterilized flows in under the radiation source and the sterilized fluid flows out above the radiation source in the channel; a first partition wall and a second partition wall are both provided in the channel, partitioning the channel into three parts, i.e. an inlet zone (A), a sterilization zone (B) and an outlet zone (C); the first partition wall is a flow guide wall, which forces the fluid to flow into the sterilization zone (B) via an opening provided at the bottom thereof; the second partition wall is a fluid level control wall, the body and bottom of which are closed against fluid and which forces the fluid passing through the sterilization zone (B) to flow out via an opening provided at the top thereof; the height at the top of the opening provided at the bottom of the flow guide wall is lower than the height at the top of the fluid level control wall, and also lower than the height at the bottom of the radiation source; the height at the top of the flow guide wall is not limited, as long as it is ensured that the fluid flowing in the inlet zone (A) would not get over the top of the flow guide wall and into the sterilization zone (B).

2. The open-channel radiation sterilization system according to claim 1, characterized in that the height at the top of the fluid level control wall is set such that the radiation source is always completely submerged in the fluid in the sterilization zone (B).

3. The open-channel radiation sterilization system according to claim 1, characterized in that the radiation source comprises UV lamps.

4. The open-channel radiation sterilization system according to claim 3, characterized in that the UV lamps are arranged parallel to the general flow direction in the channel.

5. The open-channel radiation sterilization system according to claim 3, characterized in that the UV lamps are arranged perpendicular to the general flow direction in the channel.

6. The open-channel radiation sterilization system according to claim 1, characterized in that the flow guide wall is perpendicular to, parallel to or at an angle with the general flow direction in the channel.

7. The open-channel radiation sterilization system according to claim 1, characterized in that the fluid level control wall is perpendicular to, parallel to or at an angle with the general flow direction in the channel.

8. The open-channel radiation sterilization system according to claim 1, characterized in that a plurality of groups consisting of an inlet zone (A), a sterilization zone (B) and an outlet zone (C) are provided in the channel along the general flow direction therein.

9. The open-channel radiation sterilization system according to claim 1, characterized in that the box(es) of the electrical control means is arranged in the inlet zone (A) or in the outlet zone (C), or is embedded in the channel or in the wall body of the partition walls, so as to have heat dispersed through the fluid flow.

10. The open-channel radiation sterilization system according to claim 1, characterized in that the size of the opening provided at the bottom of the flow guide wall is adjustable.

11. The open-channel radiation sterilization system according to claim 1, characterized in that the height at the top of the fluid level control wall is adjustable.
